(19) United States
(12) Patent Application Publication
(10) Pub. No.: US 2015/0216947 A1
(43) Pub. Date: Aug. 6, 2015

(54) METHOD AND COMPOSITION FOR TISSUE/CELL REPAIR

(71) Applicant: GEORGE D. PETITO, BETHLEHEM, PA (US)

(72) Inventor: GEORGE D. PETITO, BETHLEHEM, PA (US)

(21) Appl. No.: 14/547,122

(22) Filed: Nov. 18, 2014

Related U.S. Application Data
(60) Provisional application No. 61/935,073, filed on Feb. 3, 2014.

(51) Int. Cl. A61K 38/39; A61K 8/65; A61Q 19/00; A61K 45/06

(52) U.S. Cl. CPC A61K 38/39; A61K 45/06; A61K 8/65; A61Q 19/00; A61K 2800/74

(57) ABSTRACT

The method and composition for tissue/cell repair facilitates healing of damaged tissues, promoting tissue and cell growth, protecting cells and tissues, and reducing scar tissue. The composition includes hydrolyzed collagen, preferably, high molecular weight hydrolyzed collagen. The hydrolyzed collagen may be combined with native collagen and/or at least one other therapeutic agent. For example, the therapeutic agent may be a polysulfated glycosaminoglycan, a glucosamine salt, or mixtures thereof.

### CROSS-REFERENCE TO RELATED APPLICATION

This application claims the benefit of U.S. Provisional Patent Application Serial No. 61/935,073, filed Feb. 3, 2014.

### BACKGROUND OF THE INVENTION

1. Field of the Invention

The present invention relates to wound healing, and particularly to a method and composition for tissue/cell repair in animals or humans that provides for administering a composition comprising hydrolyzed collagen as the basic ingredient to promote wound healing, bacteriostasis, and scar reduction.

2. Description of the Related Art

Just as nature has provided the skin as a barrier for protection, it has also provided mechanisms for skin repair. Depending upon the nature of the injury, this repair process may take hours, days, months, or even years. Many factors determine the length of time it takes for injured skin to heal. Pathogenic contaminants may enter the body through the wound until the skin's integrity is restored. For this reason, it is desirable to heal open wounds as quickly as possible.

Open wounds in the skin are a potential gateway for infectious or contaminating material to enter the body. The skin is a protective barrier to external contaminants. When the skin is damaged with an open breach, these contaminants are free to enter the body. Once inside the body, these contaminants may have effects of varying degrees, but almost always become more difficult to treat, and consequently slow the process of healing the original wound.

In order to fight infection, wound management traditionally involves an initial cleansing of the affected area to remove any contaminants, such as dirt, clothing particles, or other debris. Damaged tissue and foreign materials are removed when necessary, and antiseptic agents are applied to sterilize the injured area. Sterile dressings are often applied, and are periodically changed to keep the injured area as clean and sterile as possible. Complex biological mechanisms occur during the healing process, such as chemical signals attracting fibroblast cells to the wound site, which ultimately generate connective structures, mainly of collagen. Endothelial cells generate new blood capillaries that nurture the new growth. Cell growth continues until the open wound is filled by forming permanent new tissue.

Traditional methods of wound healing have disadvantages, such as incomplete pigment removal, non-selective tissue destruction, and unsatisfactory cosmetic results, such as atrophic or hypertrophic scarring.

Thus, a method and composition for tissue/cell repair solving these problems is desired.

### SUMMARY OF THE INVENTION

The method and composition for tissue/cell repair facilitates healing of damaged tissues, promoting tissue and cell growth, protecting cells and tissues, and reducing scar tissue. The composition includes hydrolyzed collagen, preferably, high molecular weight hydrolyzed collagen. The hydrolyzed collagen may be combined with native collagen and/or at least one other therapeutic agent. For example, the therapeutic agent may be a polysulfated glycosaminoglycan, a glucosamine salt, or mixtures thereof.

These and other features of the present invention will become readily apparent upon further review of the following specification.

### DETAILED DESCRIPTION OF THE PREFERRED EMBODIMENTS

The present composition for tissue/cell repair includes hydrolyzed collagen, which may facilitate tissue and cell growth, as well as wound healing. Hydrolyzed collagen is a collagen hydrolysate polypeptide having a molecular weight lower than native collagen. Hydrolyzed collagen may be obtained by hydrolysis of native collagen. This may be accomplished by one of four methods: (1) alkaline hydrolysis; (2) enzymatic hydrolysis; (3) acid hydrolysis; and (4) synthetically, by fermentation. Any of these methods can be used to derive the hydrolyzed collagen from a collagen source. The collagen source can be a bovine (skin and tendon preferred), porcine, fish, avian, or a synthetic source. The hydrolyzed collagen can be derived from a combination of two or more collagen sources, e.g., a bovine source and a fish source. The fish source can include any fish. Preferably, the fish source includes salmon, tilapia, or a combination of salmon and tilapia. The types of amino acid constituents and their sequences determine the beneficial healing qualities of hydrolyzed collagen. Hydroxylysine and hydroxyproline are amino acids found only in collagen and in no other medical protein hydrolysates. Hydroxylysine is typically found in concentrations from 0.7 to 1.2 wt, % in hydrolyzed collagen.

While hydrolyzed collagen of any molecular weight may be used, the hydrolyzed collagen in the present composition is preferably high molecular weight hydrolyzed collagen, hereinafter “HMW hydrolyzed collagen,” having a molecular weight less than native collagen. For example, the HMW hydrolyzed collagen may have a molecular weight of from about 10,000 to about 300,000 Daltons, particularly from about 10,000 to about 95,000 Daltons. The present inventor has found that the cell and tissue healing properties of HMW hydrolyzed collagen, surprisingly, far exceed those of hydrolyzed collagen with lower molecular weights, i.e., less than 10,000 Daltons. In particular, HMW hydrolyzed collagen achieves bacteriostasis faster and longer than hydrolyzed collagen having a molecular weight less than 10,000 Daltons. Achieving bacteriostasis quickly and for an extended duration promotes better cell migration, and thereby accelerates recovery. Also, use of the composition may require fewer dressing changes, thereby minimizing costs for recovery. In addition, the HMW hydrolyzed collagen provides a better occlusive barrier to the injured site than hydrolyzed collagen having a molecular weight less than 10,000 Daltons. In other words, the HMW hydrolyzed collagen functions as a protective barrier and covering for forming tissues and cells, thereby further facilitating accelerated cellular repair and wound healing. Accordingly, the present composition may provide exceptional bacteriostatic and cellular repair properties.

The high molecular weight hydrolyzed collagen can be prepared by partially hydrolyzing native collagen in any suitable manner known in the art. Preferably, raw materials from one or more collagen sources are ground to a powder, enzymatically treated, fractionated, and purified to obtain high molecular weight hydrolyzed collagen. Bulk fractionation methods known in the art can be used. The raw materials can include, for example, fat, blood, tissue, and/or bone marrow from one or more collagen sources. Raw material from fish can further include, e.g., fish head and/or fins.

The present composition may include a combination of hydrolyzed collagen and native collagen. Combining native collagen with hydrolyzed collagen may enhance the bacteriostatic effects, as well as the cellular repair and wound healing properties of the composition. The different molecular weights of the native collagen and the hydrolyzed collagen in the composition may facilitate better control over absorption amount and absorption time of the composition, as well as the degradation time of the composition. For example, by varying the ratio of the native collagen to hydrolyzed collagen, various absorption rates and degradation rates may be achieved. Preferably, the composition includes a combination of HMW hydrolyzed collagen and native collagen. Soluble and/or insoluble native collagen may be used.

The composition may include about 1% by weight to about 99% by weight hydrolyzed collagen. For example, the composition may include about 10% by weight to about 85% by weight hydrolyzed collagen or about 20% by weight to about 75% by weight hydrolyzed collagen, or about 30% by weight to about 65% by weight hydrolyzed collagen. The hydrolyzed collagen is preferably HMW hydrolyzed collagen. The composition may include about 0.1% by weight to about 65% by weight of soluble or insoluble native collagen. For example, the composition may include about 2% by weight to about 45% by weight of soluble or insoluble native collagen, or about 10% by weight to about 30% by weight of soluble or insoluble native collagen. The composition may include hydrolyzed collagen cross-linked with native collagen. For example, the composition may include about 0.1% by weight to about 65% by weight insoluble or soluble native collagen crosslinked with HMW hydrolyzed collagen. Other amounts below and above these ranges may be used.

One or more additional therapeutic agents may be included in the composition to further speed the healing process, decrease scarring and increase tissue strength. Examples of suitable therapeutic agents that may be combined with the hydrolyzed collagen are glycosaminoglycans (GAGs), particularly GAGs useful for cellular repair. Antimicrobials may also be included in the composition to further enhance its bacteriostatic quality, as can antibiotics (such as tetracycline, streptomycin, and cephalosporin) and antibacterials (such as iodine, parachlorometaxylenol, and chlorhexidine gluconate or acetate). The composition may further include lipoic acid, one or more vitamins (e.g., vitamin A, vitamin B12, vitamin C, vitamin E), omega compounds or omega-3 fatty acid compounds (e.g., ALA, EPA, DHA), antioxidants (e.g., superoxide dismustase, glutathione peroxidase, glutathione reductase), and/or phytochemicals (e.g., zeaxanthin, lutein). Also, it has been established that hydrolyzed collagen used as a carrier in powder form, paste or a lyophilized foam has hemostatic qualities when combined with thrombin to improve healing of wounds.

Glycosaminoglycans (GAGs) are polysaccharides found in vertebrate and invertebrate animals. Several GAGs have been found in tissues and fluids of vertebrate animals. The known GAGs are chondroitin sulfate, keratin sulfate, dermatic sulfate, hyaluronic acid, heparin, and heparin sulfate. GAGs and collagen are the major structural elements of all animal tissue. Their synthesis is essential for proper repair, treatment, protection, and maintenance of all tissues.

A particularly preferred glycosaminoglycan is chondroitin sulfate, a polysulfated GAG. Chondroitin sulfate is a linear polymer occurring in several isomers, named for the location of the sulfate group. Chondroitin-4 sulfate is found in nasal and tracheal cartilages of bovines and porcines. It is also found in the bones, flesh, blood, skin, umbilical cord, and urine of these animals. Chondroitin-6 sulfate has been isolated from the skin, umbilical cord, and cardiac valves of the aforementioned animals. Chondroitin-6 sulfate has the same composition, but slightly different physical properties from the chondroitin-4 sulfate. These are the most common isomers used in the present composition. The polymers are also known as polysulfated glycosaminoglycans (PSGAGs), chondroitin polysulfate sodium, chondrin, sodium chondroitin polysulfate, and sodium chondroitin sulfate. For consistency, the term “chondroitin sulfate” will be recited for all chondroitin sulfate isomers throughout this specification. Chondroitin sulfate is involved in the binding of collagen, and is also directly involved in the retention of moisture in the tissue. These are both valuable chemical properties that aid the healing process.

Hydrolyzed collagen in combination with GAGs, specifically a PSGAG (such as chondroitin sulfate), can be useful for the prevention and treatment of wound diseases. The hydrolyzed collagen combines with a PSGAG to bond or adhere selectively to tissue, resulting in interference with and/or displacement of bacterial or other infectious agents. In addition, the combination product may exhibit anti-enzyme activity or the ability to inhibit enzyme activity.

The hydrolyzed collagen accelerates the healing process by allowing an injured tissue to repair itself by producing and remodeling more collagen and other proteoglycans (PGs). The building blocks for collagen production are the amino acids found in hydrolyzed collagen. Hyaluronic acid and other proteoglycans (PGs) provide the framework for collagen production to follow. The PGs hold water to provide an excellent environment for healing of the tissue to begin. When in the wound site, any unused collagen that was produced is simply degraded to the amino acid. The rate-limiting step in the production of collagen is the conversion of glucose to glucosamine for the production of hyaluronic acid and other glycosaminoglycans (GAGs).

The composition may be used to heal topical and/or internal wound sites. For example, the composition may be used prior to and after surgery to minimize cell damage and to expedite wound healing. The composition may be useful during surgery to foster separation of tissue to prevent adhesion formation. The composition may be used as a filler for a wound site and remain in the wound site as it heals, becoming part of the granulated tissue.

The composition may be useful for applications relating to cosmetic and plastic surgery, e.g., as a filler for lines and wrinkles formed in the skin.

The composition may take a physical form used in topical administration, such as a gel, spray, powder, paste, foam, film for incorporation in a dressing bandage, or a topically applied patch. The composition may take a physical form used in internal administration, such as an injectable liquid or an orally ingestible liquid.

The powder form will preferably have a moisture content of about 2-10 wt. % and a pH range of 5.5 to 6.5. The powder composition will have an ash content of less than 2.5 wt. % and an isotonic point of 5.0 to 6.5. In use, the powder composition may be the preferred physical form for use with irregularly shaped wounds. Tunnel wounds, flaps, and other non-conformative sites may be managed with the powder composition because it easily conforms to any shape wound, and may be applied by a poofer bottle or otherwise blown into difficult to reach wound sites. The powder is especially useful in wounds having a large amount of exudate, as the powder can absorb nearly 30 times its own weight. As the powder absorbs the exudate, a gel is formed, which completely fills the wound site, forming a mechanical barrier against bacterial infection. The powder does not exhibit the characteristic fly-away when being applied to the wound site, and administration is perfected due to the precise powder placement.

The gel form of the composition is especially useful in wounds with lesser amounts of exudate, in burns, and in surgical sites. Application of the gel can be dispensed through a tube, a syringe, or the reservoir in a topical patch. The gel can be made of about 1-75 wt. % HMW hydrolyzed collagen and 1-99 vol. % water. It is preferable to use about 60 wt. % collagen. The gel is formed by adding sterile water to the powder. The gel has the added advantage of adding moisture to the wound site, as well as inherent bacteriostatic properties, and stays positioned where applied.

A film form of the medicament composition may be made by mixing the powdered form with deionized water under heat at 155-175° F. Cross-linking and other agents, such as humectant, propylene glycol, sorbitol, and glycerine, may be added to the mixture. A preservative (such as benzyl alcohol or paraben) can be added. The mixture is cast on a belt liner by knife on a roll coating machine to form a liquid film, which is oven-dried. The film form can also be formed by cooling the liquid solution. These films can be used for drug or other chemical delivery, especially in dental applications. Antimicrobial and other medicinal agents can also be added to the film as needed for specific applications.

The composition may be formulated as a nutritional supplement. For example, at least one of vitamin A, vitamin C, vitamin E, vitamin B12, magnesium oxide, chelated manganese, grape seed extract, zinc, chromium picolinate, selenium, and glycosaminoglycans can be added to the composition to produce a nutrient composition for oral intake.

It is to be understood that the present invention is not limited to the embodiments described above, but encompasses any and all embodiments within the scope of the following claims.

1. A composition for tissue/cell repair, comprising:
hydrolyzed collagen,
native collagen, and
at least one antioxidant selected from the group consisting of lipoic acid, a vitamin, an omega compound, an omega-3-fatty acid compound, an antioxidant, and a phytochemical,
wherein the hydrolyzed collagen is high molecular weight collagen, the high molecular weight collagen having a molecular weight between 10,000 Daltons and 300,000 Daltons.

2. The composition according to claim 1, wherein the composition comprises:
about 1% by weight to about 99% by weight hydrolyzed collagen; and
about 0.1% to about 65% by weight native collagen.

3. (canceled)

4. The composition according to claim 2, wherein the high molecular weight collagen has a molecular weight between 10,000 Daltons and 95,000 Daltons.

5. The composition according to claim 2, wherein the native collagen comprises soluble collagen.

6. The composition according to claim 2, wherein the native collagen comprises insoluble native collagen.

7. The composition according to claim 2, further comprising a glycosaminoglycan selected from the group consisting of chondroitin sulfate, keratin sulfate, dermatic sulfate, hyaluronic acid, heparin, and heparin sulfate.

8. The composition according to claim 2, further comprising an antibiotic selected from the group consisting of tetracycline, streptomycin, and cephalosporin.

9. The composition according to claim 2, further comprising an antibacterial agent selected from the group consisting of iodine, parachlorometaxylenol, and chlorhexidine gluconate, and chlorhexidine acetate.

10. (canceled)

11. The composition according to claim 2, wherein the composition is in a topical application form selected from the group consisting of a gel, a spray, a powder, a paste, a foam and a film.

12. The composition according to claim 2, wherein the composition is in liquid form.

13. The composition according to claim 2, wherein the hydrolyzed collagen is derived from at least one collagen source selected from the group consisting of a bovine source, a porcine source, a fish source, an avian source, and a synthetic source.

14. The composition according to claim 13, wherein the fish source comprises at least one fish selected from the group consisting of salmon and tilapia.

15. A method of making a composition for tissue/cell repair, comprising:
deriving a first amount of native collagen from a collagen source;
hydrolyzing the first amount of native collagen to form a degraded collagen;
fractionating the degraded collagen to provide collagen fractions;
purifying the degraded collagen to form a high molecular weight hydrolyzed collagen having a molecular weight between 10,000 Daltons and 300,000 Daltons; and
combining the high molecular weight hydrolyzed collagen with a second amount of native collagen to form a composition for tissue/cell repair;
wherein the first amount of native collagen and the second amount of native collagen are derived from a collagen source selected from the group consisting of a bovine source, a porcine source, a fish source, an avian source, and a synthetic source.

16. The method of making a composition for tissue/cell repair according to claim 15, wherein the native collagen is derived from more than one collagen source.

17. The method of making a composition for tissue/cell repair according to claim 15, wherein the high molecular weight collagen has a molecular weight between 10,000 Daltons and 95,000 Daltons.

18. The method of making a composition for tissue/cell repair according to claim 15, wherein the hydrolyzed collagen is derived from at least one source selected from the group consisting of a bovine source, a porcine source, a fish source, an avian source, and a synthetic source.

19. A method for tissue/cell repair, comprising the step of administering a composition to a site of tissue/cell injury, the composition including between 1% by weight and 99% by weight hydrolyzed collagen, and between 0.1% by weight and 65% by weight native collagen.

20. The method for tissue/cell repair according to claim 19, wherein the hydrolyzed collagen is high molecular weight hydrolyzed collagen having a molecular weight between 10,000 Daltons and 300,000 Daltons.
